(19) United States
(12) Patent Application Publication
(10) Pub. No.: US 2009/0029901 A1
(43) Pub. Date: Jan. 29, 2009

(54) Process for Crystallizing Lactose Particles for Use in Pharmaceutical Formulations

(76) Inventor: Marian Wood-Kaczmar, Stevenage, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/815,897

(22) PCT Filed: Jan. 19, 2006
(86) PCT No.: PCT/US06/02016

§ 371 Date: May 20, 2008

Related U.S. Application Data
(60) Provisional application No. 60/651,754, filed on Feb. 10, 2005.

(51) Int. Cl. A61K 38/00; C13K 5/00; A61K 31/135; A61K 31/137; A61K 31/56

(52) U.S. Cl. 514/2; 536/123.13; 514/651; 514/653; 514/178; 514/171

(57) ABSTRACT

A process for producing a plurality of lactose particles comprises subjecting a plurality of lactose particles, to conditions such that at least a portion of smaller lactose particles detach from the plurality of the lactose particles and disperse in the liquid medium; subjecting the liquid medium to conditions sufficient to cause crystallization to occur on the smaller lactose particle surfaces to form larger lactose particles; and subjecting the liquid medium to conditions such that at least a portion of the lactose particles smaller relative to the plurality of larger lactose particles are dissolved in the liquid medium, wherein crystallization occurs on the plurality of larger lactose particles.

### FIELD OF THE INVENTION

The invention generally relates to processes for producing lactose particles.

### BACKGROUND OF THE INVENTION

In the field of inhalation therapy, it is generally desirable to employ therapeutic molecules having a particle size (i.e., diameter) in the range of 1 to 10 μm. Carrier molecules or excipients, such as lactose, for inhaled therapeutic preparations often exhibit a significantly larger diameter (e.g., 100 to 150 μm) so that they typically do not penetrate into the upper respiratory tract to the same degree as the active ingredient. However, in many instances, it is desired to use a smaller particle size for the lactose or a lactose blend having a defined ratio of coarse and fine lactose.

The lactose particle size and distribution will also, in many instances, significantly influence pharmaceutical and biological properties, such as, for example, bioavailability. For example, it is well known that coarse lactose in crystalline form has a fair flow rate and good physical stability whereas fine lactose powder, such as that produced by conventional fine grinding or milling, generally lacks good flow properties. Lactose prepared by conventional spray drying either lacks desired flow properties or contains too many large sized lactose crystals.

It is well known that one particular drawback associated with conventional means of producing pharmaceutical grade lactose relates to undesirable variations in particle size, morphology and distribution. Such production methods are particularly problematic in that they often lead to excessive and undesirable variations in the fine particle mass (“FPMass”) of pharmaceutical formulations employing such lactose. FPMass is the weight of medicament within a given dose that reaches the desired size airways to be effective.

It would be desirable to employ a process capable of producing lactose having a more consistent particle size distribution.

### SUMMARY OF THE INVENTION

In one aspect, the invention provides a process for producing a plurality of lactose particles having a specified particle size distribution. The process comprises subjecting a plurality of lactose particles, present in a liquid medium and having a plurality of smaller lactose particles on surfaces of the lactose particles, to conditions such that at least a portion of the smaller lactose particles detach from the plurality of the lactose particles and disperse in the liquid medium; subjecting the liquid medium to conditions sufficient to cause crystallization to occur on the smaller lactose particle surfaces to form a plurality of larger lactose particles therefrom, wherein a plurality of lactose particles smaller relative to the plurality of larger lactose particles are also present in the liquid medium; and subjecting the liquid medium to conditions such that at least a portion of the lactose particles smaller relative to the plurality of larger lactose particles are dissolved in the liquid medium, wherein crystallization occurs on the plurality of larger lactose particles.

These and other aspects are provided by the present invention.

### BRIEF DESCRIPTION OF THE DRAWINGS

FIG. 1 is an SEM image of a seed lactose particle having 2-3 micron fines attached thereto for use in the process of the invention.

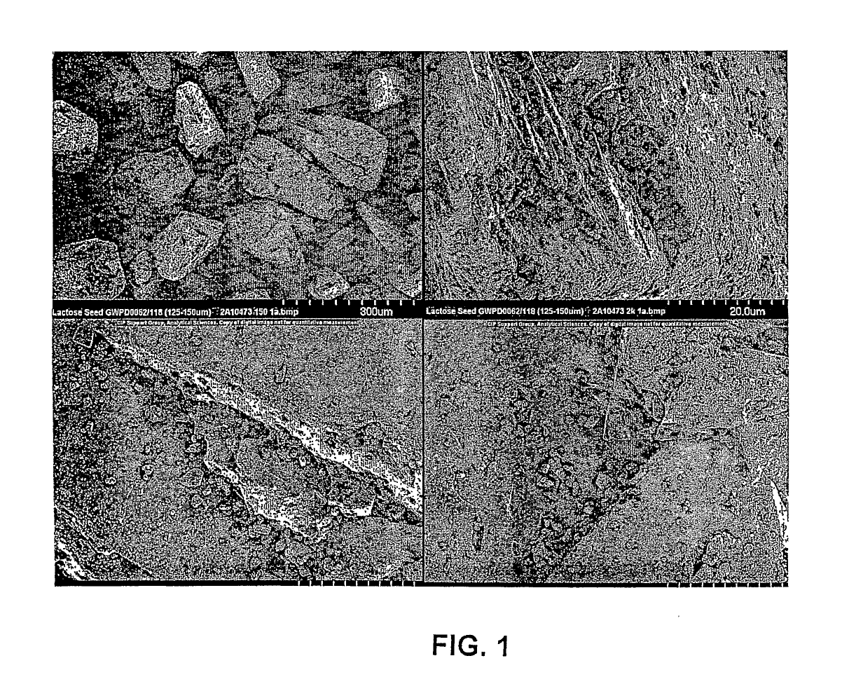

FIG. 2 is an SEM image of lactose particle formed according to the invention.

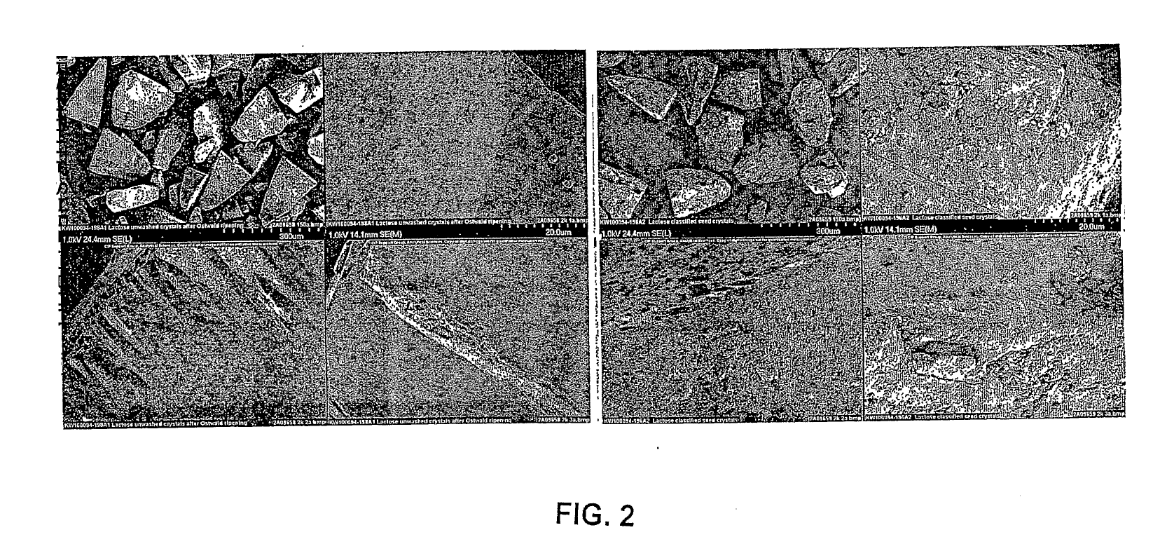

FIG. 3 is a schematic diagram of an embodiment of a lactose crystallization process employed according to the present invention.

FIGS. 4a and 4b are respectively a half-normal plot and a interaction graph illustrating the effect of process variables on lactose particle size.

FIG. 5 illustrates the particle size distributions for various lactose batches formed in accordance with the invention.

FIG. 6 illustrates gas chromatographs (GCs) for α-lactose and β-lactose for feed lactose.

FIG. 7 illustrates process control applied to a lactose crystallization process based on tomography data.

### DETAILED DESCRIPTION OF THE INVENTION

The invention will now be described with respect to the embodiments set forth herein. It should be appreciated that these embodiments are set forth to illustrate the invention, and that the invention is not limited to these embodiments. Such embodiments may or may not be practiced mutually exclusive of each other.

All publications, patents, and patent applications cited herein, whether supra or infra, are hereby incorporated herein by reference in their entirety to the same extent as if each individual publication, patent, or patent application was specifically and individually indicated to be incorporated by reference.

It must be noted that, as used in the specification and appended claims, the singular forms “a”, “an” “the” and “one” include plural referents unless the content clearly dictates otherwise.

In accordance with the present invention, the term “lactose” as used herein is to be broadly construed. As an example, lactose is intended to encompass physical, crystalline, amorphous and polymorphic forms of lactose, including, but not limited to, the stereoisomers α-lactose monohydrate and β-anhydrous lactose, as well as α-anhydrous lactose. Combinations of the above may be used. Lactose (i.e., milk sugar) is preferably obtained from cheese whey, which can be manufactured in different forms depending on the process employed. In one embodiment, the plurality of lactose particles comprise α-lactose monohydrate. In one embodiment, the plurality of lactose particles consist essentially of α-lactose monohydrate. In one embodiment, the plurality of lactose particles consist of α-lactose monohydrate. In one embodiment, the α-lactose monohydrate may have an anomeric purity of at least 97 percent. As used herein, the term “particle” is to be broadly interpreted to encompass those of various shapes, sizes, and/or textures which can include those that may have varying degrees of irregularities, disuniformities, etc. or which may possess regular and/or uniform properties.

In one embodiment, the liquid medium is an aqueous medium, i.e., more than 40 percent by weight of the medium is water. In one embodiment, a saturated lactose solution may include 47.6% wt/wt of water. Co-solvents may be employed including, without limitation, ethanol and acetone. In one embodiment, for example, the medium may include 45% wt/wt wt ethanol/water. In one embodiment, the medium may include 45% wt/wt acetone/water. Particle sizes of below 10 microns may be achieved with the above cosolvent mixtures. The term “water” is to be broadly interpreted to encompass tap water, treated (e.g., distilled) water, purified water, as well as other types of water. The liquid medium may also be employed as an organic medium. One example of an organic solvent that may be used is dimethyl sulphoxide. Mixture of any of the above aqueous and organic mediums can be employed. The liquid medium utilized in accordance with the present invention can also optionally encompass a wide range of additives and additional components such as, without limitation, surfactants, buffers, wetting agents, and the like.

The lactose particles employed (i.e., seed material) in the process of the invention may have various size distributions. For example, in one embodiment, the lactose particles may have a median diameter (D-50) ranging from, at a lower end, about 70, 80, or 90 microns to, at a higher end, about 100, 110, 120, or 130 microns.

The smaller lactose particles present on the surfaces of the lactose particles are present in various configurations. As an example, the term “on” can be interpreted to mean that the smaller particles can be attracted to the surface of the lactose particles in different manners. For example, the larger particles may be coated with the smaller particles.

In various embodiments, the smaller lactose particles present on the surfaces of the lactose particles may be present in various sizes. As an example, the plurality of smaller particles may have a median diameter (D-50) ranging from about 1 micron to about 3 microns, as obtained from SEM images.

In accordance with the invention, at least a portion of the smaller lactose particles detach from the lactose particles. In one embodiment, the smaller lactose particles disperse so as to form a homogeneous dispersion in the liquid medium.

The step of subjecting a plurality of lactose particles to conditions such at least a portion of the smaller lactose particles detach from plurality of lactose particles may occur under various conditions. For example, in one embodiment, such a step may occur such that the liquid medium may have a temperature ranging from about 50° C. to about 70° C. In one embodiment, the liquid medium may have a temperature of 50° C. Although not intending to be bound by theory, interactions between temperature and seed size suggest that at higher temperatures the formed lactose is smaller whereas seeding temperature alone does not have an effect on particle size. Micronized seed does not show the same effect suggesting that small particles are associated with the larger seed and either being chipped off by attrition or being detached from the surface by the action of the liquid medium. Again, not intending to be bound by theory, the PSD of the product tends higher at lower temperatures so that attrition is not the cause and attachment of fine particles to the seed surface was the most likely explanation. This was confirmed by SEM. The conclusion is that more particles tend to be detached from the surface at higher temperatures. The optimum temperature range has not been established, and at temperatures lower than 50° C. spontaneous nucleation might occur and at temperatures higher than 70° C. fine particle seeds might dissolve.

Additionally, in one embodiment, the liquid medium may have a pH ranging from about 3.0 to about 4.0.

Moreover, in one embodiment, the liquid medium is supersaturated with lactose. For the purposes of the invention, the term “supersaturated” is defined as the actual concentration of solute (C) in solvent minus the solubility of the solute in that solvent (C*) at a constant temperature and solution composition. Supersaturation may be expressed as shown below

S=C−C*

As an example, in one embodiment, the supersaturation of the lactose solutions at normal crystallization conditions used at 50° C., from the equation above is 62 g/100 g water and 27 g/100 g water at 70° C.

As set forth hereinabove, the invention also encompasses the step of subjecting the liquid medium to conditions sufficient to cause crystallization to occur on the smaller lactose particles to form a plurality of larger lactose particles therefrom. The plurality of larger lactose particles may encompass a number of sizes. For example, in one embodiment, the plurality of larger lactose particles may have a median diameter (D-50) ranging from about, at a lower end, about 20, 30, 40, 50, or 60 microns to, at a higher end, about 70, 80, 90, 100, 110, or 130 microns. The step of subjecting the liquid medium to conditions sufficient to cause crystallization to occur on the smaller lactose particles may take place under various conditions. For example, in one embodiment, such a step may occur such that the liquid medium may have a temperature ranging from, at a lower end, about 20, 25, 30 or 35° C. to, at a higher end, about 35, 40, 45, or 50° C. In one embodiment, the liquid medium has a temperature of 50° C. In one embodiment, the liquid medium may have a pH ranging from about 3 to about 4.

The step of subjecting the liquid medium to conditions such that at least a portion of the lactose particles smaller relative to the plurality of larger lactose particles are dissolved in the liquid medium, wherein crystallization occurs on the plurality of larger lactose particles may encompass various embodiments. For example, in one embodiment, the lactose particles formed as a result of the crystallization may have a median diameter (D-50) ranging from, at a lower end, about 20, 30, 40, 50, 60, 70 or 80 microns to, at a higher end, about 70, 80, 90, 100, 110, 120 or 130 microns.

In one embodiment, the resulting crystallized lactose particles are substantially free of surface defects. More specifically, the resulting crystallized lactose particles may be present as smooth regular tomahawks.

In conjunction with the process of the invention, other procedures known in the art can be employed which are often associated with crystallization processes. Examples of such procedures include, without limitation, cleaning and sanitization, crystallization vessel pre-wash, and inter-batch cleaning.

In other aspects, the invention may encompass pharmaceutical formulations formed by the processes, as well as inhalation devices including such formulations. Medicaments, for the purposes of the invention, include a variety of pharmaceutically active ingredients, such as, for example, those which are useful in inhalation therapy. In general, the term “medicament” is to be broadly construed and include, without limitation, actives, drugs and bioactive agents, as well as biopharmaceuticals. Various embodiments may include medicament present in micronized form. Appropriate medicaments may thus be selected from, for example, analgesics, (e.g., codeine, dihydromorphine, ergotamine, fentanyl or morphine); anginal preparations, (e.g., diltiazem); antiallergics, e.g., cromoglicate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., beclometasone dipropionate, fluticasone propionate, flunisolide, budesonide, rofleponide, mometasone furoate, ciclesonide, triamcinolone acetonide, 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester), (6α,11β,16α,17α)-6,9-difluoro-17-{[(fluoromethyl)thio]carbonyl}-1′-hydroxy-16-methyl-3-oxoandrosta-1,4-dien-17-yl 2-furoate, and (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate); antitussives, (e.g., noscapine); bronchodilators, e.g., albuterol (e.g. as sulphate), salmeterol (e.g. as xinafoate), ephedrine, adrenaline, fenoterol (e.g as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-(henylethoxy)propyl]sulfonyl]ethyl]-amino]ethyl-2(3H)-benzothiazolone), 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, and 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one; diuretics, (e.g., amiloride; anticholinergics, e.g., ipratropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament. It will be further clear to a person skilled in the art that where appropriate, the medicaments may be used in the form of a pure isomer, for example, R-salbutamol or RR-formoterol.

Particular medicaments for administration using pharmaceutical formulations in accordance with the invention include anti-allergics, bronchodilators, beta agonists (e.g., long-acting beta agonists), and anti-inflammatory steroids of use in the treatment of respiratory conditions as defined herein by inhalation therapy, for example cromoglicate (e.g. as the sodium salt), salbutamol (e.g. as the free base or the sulphate salt), salmeterol (e.g. as the xinafoate salt), bitolterol, formoterol (e.g. as the fumarate salt), terbutaline (e.g. as the sulphate salt), reproterol (e.g. as the hydrochloride salt), a beclometasone ester (e.g. the dipropionate), a fluticasone ester (e.g. the propionate), a mometasone ester (e.g., the furoate), budesonide, dexamethasone, flunisolide, triamcinolone, tripredane, (22R)-6α,9α-difluoro-11β,21-dihydroxy-16α,17α-propylmethylenedioxy-4-pregnen-3,20-dione. Medicaments useful in erectile dysfunction treatment (e.g., PDE-V inhibitors such as vardenafil hydrochloride, along with alprostadil and sildenafil citrate) may also be employed. It should be understood that the medicaments that may be used in conjunction with the inhaler are not limited to those described herein.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more medicaments. Formulations containing two active ingredients are known for the treatment and/or prophylaxis of respiratory disorders such as asthma and COPD, for example, formoterol (e.g. as the fumarate) and budesonide, salmeterol (e.g. as the xinafoate salt) and fluticasone (e.g. as the propionate ester), salbutamol (e.g. as free base or sulphate salt) and beclometasone (as the dipropionate ester) are preferred.

In one embodiment, a particular combination that may be employed is a combination of a beta agonist (e.g., a long-acting beta agonist) and an anti-inflammatory steroid. One embodiment encompasses a combination of fluticasone propionate and salmeterol, or a salt thereof (particularly the xinafoate salt). The ratio of salmeterol to fluticasone propionate in the formulations according to the present invention is preferably within the range 4:1 to 1:20. The two drugs may be administered in various manners, simultaneously, sequentially, or separately, in the same or different ratios. In various embodiments, each metered dose or actuation of the inhaler will typically contain from 25 μm to 100 μm of salmeterol and from 25 μm to 500 μm of fluticasone propionate. The pharmaceutical formulation may be administered as a formulation according to various occurrences per day. In one embodiment, the pharmaceutical formulation is administered twice daily.

Embodiments of specific medicament combinations that may be used in various pharmaceutical formulations are as follows:

1) fluticasone propionate 100 μm/salmeterol 50 μm

2) fluticasone propionate 250 μm/salmeterol 50 μm

3) fluticasone propionate 500 μm/salmeterol 50 μm

In various embodiments, the pharmaceutical formulations may be present in the form of various inhalable formulations. In one embodiment, the pharmaceutical formulation is present in the form of a dry powder formulation, the formulation of such may be carried out according to known techniques. Dry powder formulations for topical delivery to the lung by inhalation may, for example, be presented in capsules and cartridges of for example gelatine, or blisters of for example laminated aluminum foil, for use in an inhaler or insufflator. Powder blend formulations generally contain a powder mix for inhalation of the compound of the invention and a suitable powder base which includes lactose and, optionally, at least one additional excipient (e.g., carrier, diluent, etc.). In various embodiments, each capsule or cartridge may generally contain between 20 μm and 10 mg of the at least one medicament. In one embodiment, the formulation may be formed into particles comprising at least one medicament, and excipient material(s), such as by co-precipitation or coating. When employed as a dry powder, packaging of the formulation may be suitable for unit dose or multi-dose delivery. In the case of multi-dose delivery, the formulation can be pre-metered (e.g., as in Diskus®, see GB 2242134/U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g. as in Turbuhaler, see EP 69715, or in the devices described in U.S. Pat. No. 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet hermetically but peelably sealed thereto to define a plurality of containers, each container having therein an inhalable formulation containing the at least one medicament, the lactose, optionally with other excipients. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. The lid sheet may preferably be peeled from the base sheet in a longitudinal direction from a first end of the base sheet.

In one embodiment, the formulations may be employed in or as suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Such formulations may be delivered via a pressurized inhaler, e.g., a Metered Dose Inhaler (MDI). Exemplary MDIs typically include canisters suitable for delivering the pharmaceutical formulations. Canisters generally comprise a container capable of withstanding the vapour pressure of the propellant used such as a plastic or plastic-coated glass bottle or preferably a metal can, for example an aluminum can which may optionally be anodised, lacquer-coated and/or plastic-coated, which container is closed with a metering valve. Aluminum cans which have their inner surfaces coated with a fluorocarbon polymer are particularly preferred. Such polymers can be made of multiples of the following monomeric units: tetrafluoroethylene (PTFE), fluorinated ethylene propylene (FEP), perfluoroalkoxyalkane (PFA), ethylene tetrafluoroethylene (EFTE), vinyidienefluoride (PVDF), and chlorinated ethylene tetrafluoroethylene. Embodiments of coatings used on all or part of the internal surfaces of an MDI are set forth in U.S. Pat. Nos. 6,143,277; 6,511,653; 6,253,762; 6,532,955; and 6,546,928.

MDIs may also include metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK356) and 3M-Neotechnic Ltd, UK (e.g. Spraymiser™). Embodiments of metering valves are set forth in U.S. Pat. Nos. 6,170,717; 6,315,173; and 6,318,603.

In various embodiments, the MDIs may also be used in conjunction with other structures such as, without limitation, overwrap packages for storing and containing the MDIs, including those described in U.S. Pat. No. 6,390,291, as well as dose counter units such as, but not limited to, those described in U.S. Pat. Nos. 6,360,739 and 6,431,168.

In addition to the above, the pharmaceutical formulations can be employed in capsules, sachets, tablet buccals, lozenges, papers, or other container. Moreover, the formulations can be in the form of tablets, pills, powders, elixirs, suspensions, emulsions, solutions, syrups, capsules (such as, for example, soft and hard gelatin capsules), suppositories, sterile injectable solutions, and sterile packaged powders. Excipients, carriers, diluents, and the like may be optionally employed.

The pharmaceutical formulation formed by the processes of the invention may be used in the treatment of a number of respiratory disorders. Such respiratory conditions include, without limitation, diseases and conditions associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis). Such treatment is carried out by delivering medicament to a mammal. Accordingly, and in view of the above, in another aspect, the invention provides a method for the treatment of a respiratory disorder comprising the step of administering a pharmaceutically effective amount of a pharmaceutical formulation to a mammal such as, for example, a human. For the purposes of the invention, the term “pharmaceutically effective amount” is to be broadly interpreted and encompass the treatment of the disorder. In one embodiment, the administration is carried out via an inhalation device described herein. In one embodiment, the administration is carried out by nasal or oral inhalation.

The following examples are intended to illustrate the invention, and do not limit the scope of the invention as defined by the claims.

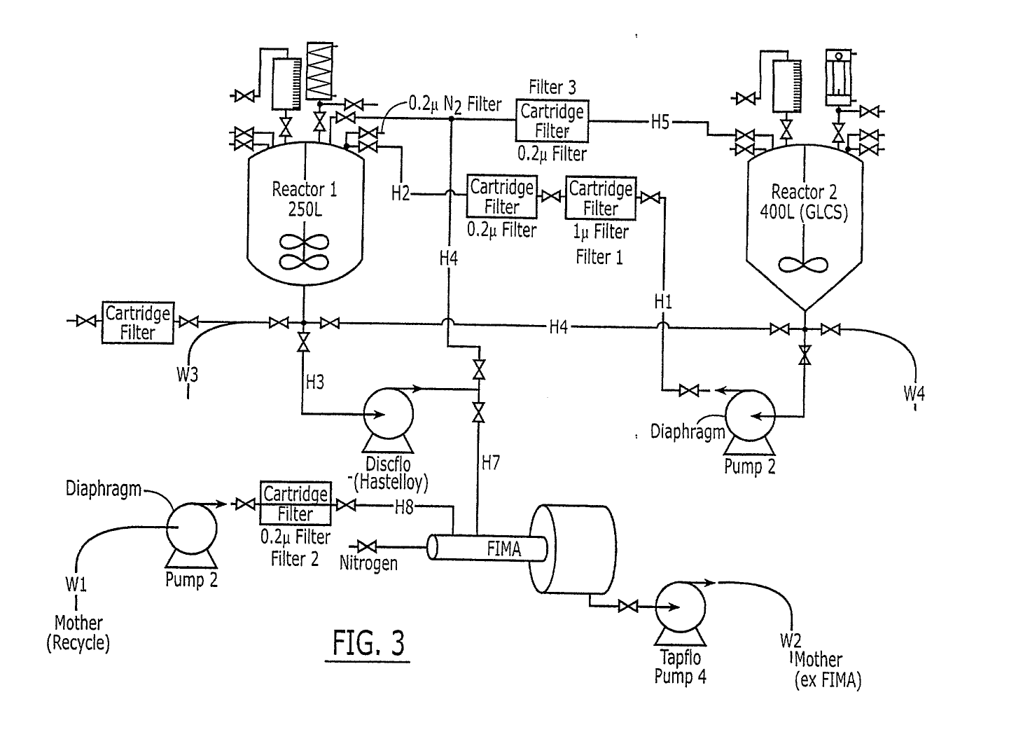

Table 1 sets forth equipment employed in the crystallization embodiments illustrated in the Examples. The crystallization process configuration is set forth in FIG. 3.

TABLE 1
Crystallization Equipment List

| Item | Ref. | Equipment | Comments |
|---|---|---|---|
| 1. | REACTOR 1 | 250 L glass lined reactor, | Crystallizer (all N₂ to this vessel to pass |
| | | dish bottom, twin flight | through 0.2μ bacterialm retentive filter) |
| | | retreat blade impeller | |
| 2. | REACTOR 2 | 400 L hastelloy reactor | Used for preparing lactose solution and |
| | | | as reservoir for hot water to clean |
| | | | REACTOR 1 and recycle loop. |
| 3. | FILTER A | Pall in-line cartridge filter | 0.2 μm bacterial retentive cartridge |
| 4. | FILTER 1 | Stainless steel cartridge | 1 μm cartridge, encapsulated ‘0’ rings |
| | | filter | |
| 5. | FILTER 2 | Stainless steel Dominic | Fitted to FIMA. |
| | | Hunter filter | 0.2 μm bacterial retentive cartridge, |
| | | | encapsulated ‘o’ rings. |
| 6. | FILTER 3 | Stainless steel Dominic | Fitted with 0.2 μm bacterial retentive |
| | | Hunter filter | cartridge, encapsulated ‘o’ rings. |
| 7. | PUMP 1 | Diaphragm pump | For recycle of mother liquors |
| 8. | PUMP 2 | Diaphragm pump | For transfer from REACTOR 2 to |
| | | | REACTOR 1 |
| 9. | PUMP 3 | Discflo pump | Connected to REACTOR 1 outlet and |
| | | | inlet via stainless pipework. |
| 10. | PUMP 4 | Tapflo pump | For FIMA centrifuge/drier |
| 11. | FILTER 4 | Small GAF filter | 1 μm nominal bag. for nitrogen supply |
| | | | line to FIMA |
| 12. | DRIER 1 | FIMA centrifuge drier | Used only for isolation of wet solid |
| | | (fill vol. 37 L, surface area | |
| | | 0.37 m²) | |
| 13. | FILTER 4 | Nitrogen filter | Fitted to REACTOR 1 outlet |
| 14. | DRIER 2 | Bolz dryer (30 L capacity) | Stainless steel |

### EXAMPLE 1

### Crystallization Process Description

### Process Description

Charge 160 kg (159.31 kg) of α-lactose monohydrate to REACTOR 2.
Charge 112 L of process water to REACTOR 2.
Start agitator in REACTOR 2 and run at 100 (80) rpm.
Heat the mixture in REACTOR 2 to 100° C. to dissolve the solid
Start agitator in REACTOR 1 and run at 50 rpm.
Transfer the hot solution in REACTOR 2 to REACTOR 1 via a 1.0 μm and 0.2 μm filter assembly.
Charge 32 L (42 L) of process water to REACTOR 2.
Heat the water in REACTOR 2 to 90° C.
Transfer the water in REACTOR 2 to REACTOR 1 via the 1.0 μm and 0.2 μm filter assembly.
Purge the vessel headspace in REACTOR 1 with nitrogen and maintain a slight positive pressure within the vessel throughout the crystallization.
Adjust the temperature of the solution in REACTOR 1 to 85° C. using only the bottom jacket.
Cool the solution to 50° C.-53° C.
When the temperature in REACTOR 1 is stable at 50-53° C. (actual seeding temperature 53.2° C.). add 8.0 g of sieved seed (125-150 μm)
Follow the cooling profile for the next 7 operations controlling the temperature on the bottom jacket.
Hold the temperature at 50° C.-53° C. for approx. 2 hours.
Cool the mixture to 44° C.-47° C. over approx. 1 hour.
Hold the mixture at 44° C.-47° C. for approx. 2 hours.
Cool the mixture to 20° C.-23° C. over approx. 5 hours.
Heat the mixture to 60° C.-63° C.
Hold the temperature at 60° C.-63° C. for approx. 2 hours.
Cool the mixture to 20° C.-23° C. over approx. 6 hours.
Start the discflo pump and re-circulate the slurry back into REACTOR 1 during the isolation of the solid.
Isolate the solid in 4-6 drops in the FIMA centrifuge.
   Unload the wet solid into polyethene bags and store the material at 0° C.-4° C. until it can be dried.
   (Wet product was stored at 0° C.-4° C. for no longer than 24 hours before drying. Wet batches were stored at 0-4° C. for up to 6 days before drying).
Wet material charged into the Bolz dryer and the headspace purged with nitrogen.
Nitrogen purge rate set to 0.5 L/min
The jacket set point temperature was set to 75° C.
The solid became dried after approximately 4 hours until the temperature and pressure had stabilized. However, actual drying times were variable ranging from 7-12 hours depending on shift changeovers, problems etc. It was determined that the preferred drying time should be set at 4-6 hours.
The temperature of the jacket was reduced to 40° C. and drying continued until the temperature had stabilised.
The contents were cooled and the product unloaded.

### Packaging and Storage

The dry solid was packed in double wrapped in food grade plastic bags and stored in plastic kegs at ambient temperature (20° C.) in the chemical intermediates store.

### EXAMPLE 2

### Crystallization of Lactose from Water

This example represents a summary of the laboratory work conducted in accordance with Example 1. The objective of this example was to produce crystalline lactose having a particle size distribution (PSD) and a low fines level.

An experiment was performed on the crystallization of lactose from water to identify the critical variables of the process. Four variables were chosen that would be independent of scale. Table 2 lists such variables.

TABLE 2

| Variable | Low value | High value | Mid point |
|---|---|---|---|
| Seed size | 5 μm | 150 μm | 77.5 |
| Seed quantity | 0.01% (6.6 mg) | 0.1% (33 mg) | 0.055% (19.8 mg) |
| Cooling time | 10 hr | 30 hr | 20 hr |
| Seeding temperature | 50 C° C. | 70° C. | 60° C. |
| Run | 1 | 2 | 3 |

Three percentile responses (D-10, D-50 and D-90) were chosen to describe the size distribution. The values were measured using a Lasentec S400 FBRM mini probe made commercially available by Mettler Toledo in Columbus, Ohio.

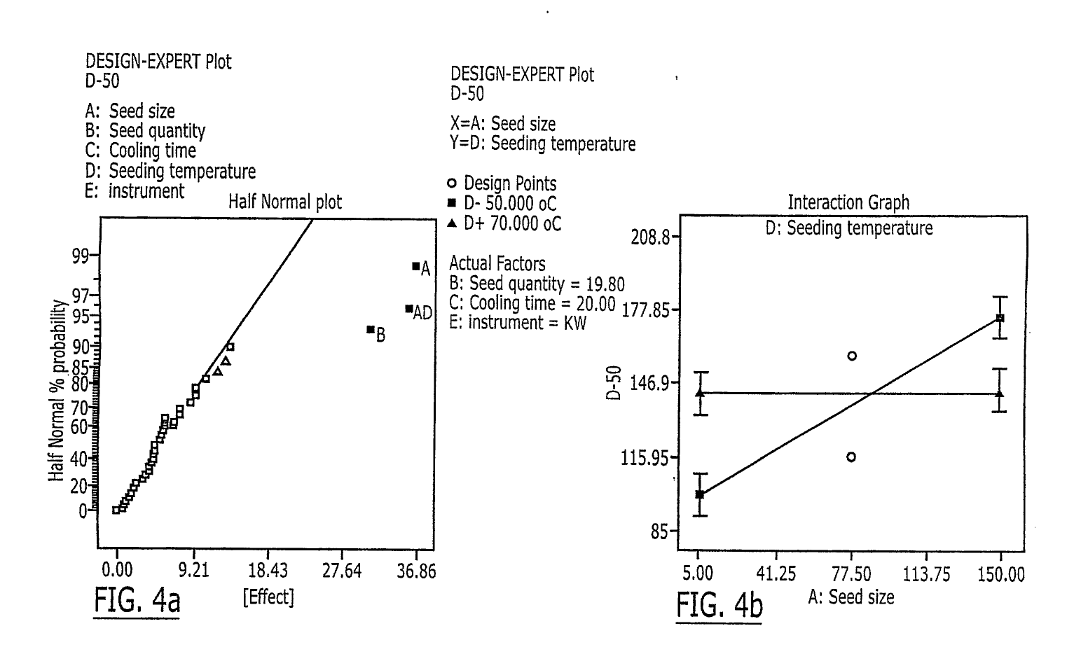

The results for the D-50 percentile values from the DoE experiments are shown in FIGS. 4a and 4b. Other responses show similar effects.

The results show the critical effect of seed size and seed quantity but also show an interaction between seed size and temperature. Surprisingly, cooling time shows little if any affect. Not intending to be bound by theory, this is probably due to the de-supersaturation rate being faster than the cooling rate and hence remains relatively constant for both cooling times.

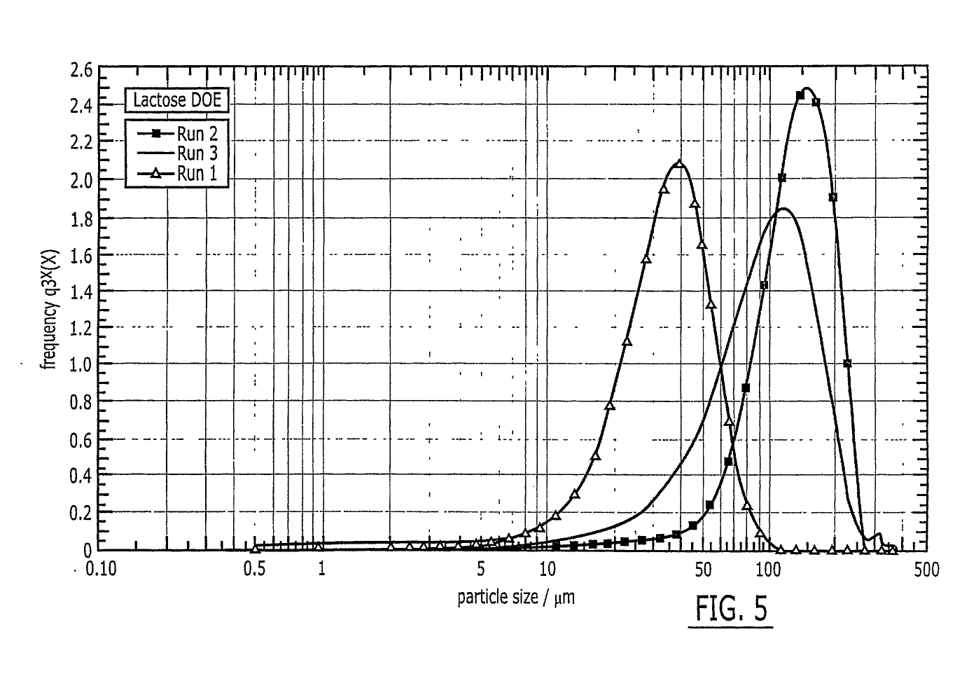

The experiments had produced a wide range of particle sizes shown by the Sympatec PSD (“particle size distribution”) on selected batches as shown in FIG. 5.

Data tend to show an interaction between temperature and seed size that suggests that at higher temperatures the product size is smaller whereas seeding temperature alone does not have an effect on particle size. Not intending to be bound by theory, it was this observation that suggested that we had a different seeding mechanism for the larger seed. The interaction graph shows that at lower temperature the increase in particle size is higher than would be expected for a conventional seeding mechanism. High temperature seeding with 5 μm micronised material gives product size that is larger than that produced at lower seeding temperature. Not intending to be bound by theory, this may be due to some microfine material (<1 μm) dissolving at the higher temperature leaving fewer seeds available for nucleation. High temperature seeding with large seed gives product size that is smaller than the corresponding low temperature seeding. Again, not intending to be bound by theory, this suggests that small particles are associated with the larger seed and either being chipped off by attrition or being detached from the surface by the action of the liquid medium and the extent of detachment is higher at higher temperature. The microfine particles are seemingly bound more strongly or are dissolved at higher temperature and so will not play any part in the nucleation. The PSD of the product is higher at lower temperatures so that attrition is not the cause and attachment of fine particles to the seed surface was the most likely explanation. This was confirmed by SEM.

One of the benefits from the invention is that the dispersion of the seeding throughout the mixture is better and because the large seed is prepared by sieving the size control of the seed is also improved.

Although not intending to be bound by theory, seeding the lactose at 50° C. with 0.02% (input wt) of classified seed (15 mm) is believed to give the largest particle size (D-10 75 mm, D-50 133 mm, D-90 204 mm). Again, not intending to be bound by theory, seeding at 50° C. with 0.1% of micronized seed is believed to give a small PSD (D-10 16 mm, D-50 34 mm, D-90 57 mm).

Further improvements to PSD were believed to be achieved by high temperature Ostwald ripening. The crystallized slurries were re-heated to 60° C. for 3 hours and slowly cooled to 20° C. The chord length distribution (CLD) measured by Lasentec FBRM show a marked shift to the larger size ranges with a considerable reduction of fine particles. Microscopic examination of the isolated crystalline solid before and after Ostwald ripening shows a significant increase in size with few fine particles present.

Application of a seeding regime using a small quantity of classified seed coupled with high temperature Ostwald ripening is believed to provide large lactose crystals with few fines, that are believed to be well-suited for secondary processing.

### EXAMPLE 3

### Process Summary

Process water (0.7 vol) and Lactose mono hydrate, are heated to 100±5° C. and stirred for ca. 30 minutes until complete dissolution is achieved then the solution is cooled to 90±2° C. It is then passed through a 0.2 m filter and the filter rinsed with process water (0.2 vol) at 90±2° C. The solution is cooled to 50±2° C., seed crystals (0.00005 wt) are added then the solution held at 50±2° C. for 2 hours. The resultant slurry is then cooled to 45±2° C. over 1 hour then held at 45±2° C. for 2 hours. The slurry is further cooled to 20±2° C. over 5 hours. The mixture is heated to 60±2° C. then held at that temperature for 2 hours then cooled to 20±2° C. over 8 hours then a sample removed for analysis. The solid is then isolated portion wise in a centrifuge and dried with nitrogen at 100±5° C., then aged for at least 30 minutes with nitrogen at 40±5° C. then cooled to <30° C. and off loaded.

### EXAMPLE 4

### Crystallization Procedure

In this example, temperature is controlled throughout crystallization. Crystallization is done under a nitrogen head-space providing a small positive pressure differential from the interior of the crystallizer to the external environment.

Initially the temperature of the lactose solution is at 90° C., it is then cooled to 50-53° C., the seed is added, and the temperature is reduced to 45° C. and on to 20° C. Microbial numbers added from the seed are likely to be minimal as the total weight of seed is only around 8 g. The outside surfaces of the charge port on the crystallizer were sanitized by spraying with a sanitizing spray and prior to charging seed, they could however survive these temperatures and possibly increase in numbers. The seed should be added with as little further environmental contamination as possible.

After 5 h at 20° C., the temperature is raised to 60° C. for 2 hours. This 60° C. hold period should inactivate any vegetative microbial contamination that may have arisen in earlier stages of the process. Temperatures should be monitored, particularly during the 60° C. hold period.

Thereafter the slurry is held at 20° C. This hold may last for up to 2 days. This is a period of serious risk. Any contaminants which may have survived earlier anti-microbial factors, any contaminants from the activated valve at the base of the crystallizer, and any contaminants which may have survived or grown in the gas line downstream of the bacteria-retentive filter may potentially increase in numbers.

### Drying

Drying is done under minimal microbiological control, and the resultant lactose is dried under a stream of nitrogen at 10° C. The lactose was dried in a BOLZ drier under nitrogen pressure of 950-100 mbarg. The nitrogen was not heated but the solid was heated by circulating water at 75-80° C. through a BOLZ jacket. A dryer manufactured by FIMA proved unsuitable.

### EXAMPLE 5

### Crystallization Procedure

A lactose crystallization is carried out according to the following procedure:

Lactose is charged to vessel REACTOR 2 and process water added.
The mixture is heated to 100° C. to dissolve the solid.
The solution is transferred into REACTOR 1 via the 1 μm and 0.2 μm filters and flushed through with hot process water.
During the crystallization, vessel REACTOR 1 is kept under positive nitrogen pressure throughout.
Prior to addition of the seed to the crystallizer the manway is sprayed with sanitising spray and the operators are required to wear clean disposable overall suits, sterile gloves and masks while adding the seed.

### EXAMPLE 6

### Solid Isolation Procedure

Centrifugation is strongly preferred for the process to enable efficient de-liquoring to approx. 5% LOD. Vacuum filtration will only reduce moisture to 10-12%. At this level the β-anomer content of the dried material will increase to greater than 3% (limit 3%).

Isolation of the solid was achieved using a FIMA centrifuge/dryer. The size of the FIMA necessitated isolation of the 100 kg batches in 5-6 drops of 15-20 kg per drop. The nature of the lactose solid leads to fast de-liquoring, uneven distribution during isolation and makes the solid difficult to dislodge from the FIMA drum after de-liquoring. Fluidised bed drying was inefficient and caused severe caking of the undislodged solid

During the isolation step, it was found that once the bottom mushroom valve had been opened, accumulation of solid around the convoluted PTFE sleeve below the mushroom valve prevented the valve from closing. Any slurry remaining in transfer pipes settled causing blockage in the lines. To overcome this problem a re-circulation loop was added to keep slurry moving during isolation. The circulating pump can damage the crystals and is not recommended (if pumping of the slurry is unavoidable then a pump designed for pumping fragile materials should be used).
The recommended isolation process would be to use a basket centrifuge large enough to accommodate the whole batch in one drop
The centrifuge should be fitted with a steam in place device for sanitizing the equipment prior to isolation
Slurry transfer lines should be sanitized before use. As an example, circulation of dilute sodium hypochlorite solution through the slurry lines followed hot water for at least 30 minutes immediately before isolation should be employed. Steam sterilization should be the method of choice for sanitising transfer lines.

### EXAMPLE 7

### Drying Procedure

In this example, a Bolz dryer was used successfully for drying the lactose drops from each of the batches at 60° C. This may be the method of choice for this product, although a higher drying temperature of 90-100° C. may be preferable.

Fluidised bed drying in the FIMA centrifuge/dryer was partially successful as described above. Fluidised bed drying is the method of choice by lactose suppliers; however the amount of crystal breakage of this drying method has not been determined.
The preferred dryer for this process a Bolz drier. Solid must be gently agitated at all times during drying to prevent caking and attrition should be minimised.
Steam sterilization of the dryer should be considered for routine processing.

### EXAMPLE 8

### Crystallization Results

The following batches were crystallized according to procedure set forth herein. Results are set forth in Tables 4.

TABLE 4

| | Wet | Dry | | | | | Percent |
|---|---|---|---|---|---|---|---|
| Batch | Weight | Weight | Dryer | D-10 | D-50 | D-90 | Water |
| 1 | 26.2 | 22.8 | Bolz | 79 | 165 | 280 | 5.3 |
| 2 | 18.9 | 17.0 | Bolz | 82 | 174 | 308 | 5.0 |
| 3 | 16.4 | 16.1 | Bolz | 85 | 178 | 317 | 5.0 |
| 4 | 15.6 | 14.6 | Bolz | 88 | 188 | 339 | 5.1 |
| 5 | 17.7 | 16.9 | Bolz | 69 | 168 | 299 | 5.1 |
| 6 | 23.1 | 21.7 | Bolz | 76 | 171 | 300 | 5.1 |
| | 117.9 | 109.2 | | | | | |

Percentage of β-anomer blend of drops 1.4

| | | | | | | | |
|---|---|---|---|---|---|---|---|
| 7 | 16.0 | 14.9 | Bolz | 87 | 176 | 311 | 5.0 |
| 8 | 25.7 | 22.1 | Bolz | 73 | 152 | 262 | 5.0 |
| 9 | 28.9 | 22.0 | Bolz | 79 | 162 | 271 | 5.0 |
| 10 | 17.9 | 23.8 | Bolz | 77 | 160 | 273 | 5.1 |
| 11 | 15.6 | 15.5 | Bolz | 81 | 165 | 296 | 4.9 |
| 12 | 13.5 | 12.9 | Bolz | 88 | 185 | 336 | 5.0 |
| | 117.5 | 111.3 | | | | | |
| 13 | 22.5 | 21.2 | Bolz | 78 | 161 | 274 | 5.1 |
| 14 | 28.7 | 25.8 | Bolz | 77 | 162 | 278 | 4.7 |
| 15 | 23.4 | 22.6 | Bolz | 80 | 167 | 288 | 4.9 |
| 16 | 20.3 | 20.6 | Bolz | 77 | 165 | 295 | 5.0 |
| 17 | 17.9 | 17.1 | Bolz | 82 | 171 | 311 | 4.8 |
| | 112.8 | 107.3 | | | | | |
| Totals | 348.2 | 327.7 | | | | | |

TABLE 5

| Batch | D-10 | D-50 | D-90 |
|---|---|---|---|
| A (blend) | 68.3 | 167.9 | 312.7 |
| B (blend) | 83.2 | 177.7 | 306.4 |
| C (blend) | 82.2 | 184.4 | 330.9 |

Table 5 represents D-10, D-50 and D-90 values for blended samples. More particularly, A (blend) represents a blend of the individual dry weights of batches 1-6, listed in Table 4. B (blend) represents a blend of the individual dry weights of batches 7-12, listed in Table 4. C (blend) represents a blend of the individual dry weights of batches 13-17, listed in Table 4. All batches listed in Table 4 were isolated and dried separately, and were synthesized under the same process conditions.

PSD results were obtained by employing a Sympatec HELOS Laser Diffraction method described in Example 11.

Karl Fisher results for all sub batches from Batches 1-17 are presented in Table 4 above. The Karl Fisher method employed is known in the art, as well as the water analysis method.

### EXAMPLE 9

### Assay and Anomer Ratio Analysis

Sub lots of each batch were blended to give a representative blend of the batch and submitted for chemical analysis. The analytical results for anomer content is described herein. The procedure that was used to make the blends for anomer content was that samples from each batch were taken that were proportional to the batch weights and mixed in a container by shaking and manual mixing.

### EXAMPLE 11

### Particle Size Analysis Procedure

This procedure was used in conjunction with batches 1-6, 7-12 and 13-17 described in Example 8 according to known procedure. All samples were measured in triplicate.

All sample preparations are to be carried out in a Class 2 safety cabinet with operators wearing gloves and eye protection in accordance with COSHH assessment for handling the relevant drug substance under analysis within Pharmacy Division and the company COSHH codes of practice and the local safe working practice of documents.

Reference: COSHH/01/06

### Instrumentation

### Sympatec HELOS Laser Diffraction, Serial Number: H0643.-Biomax:064263

### Vibri Feeder Serial Number: 528 Biomax:0642266

### Method

### Lens: R5 Pressure: 1.5 bar Feed Rate: 85%

Reference measurement: 10 s

### Time Base: 100 ms

Trigger 0 s after opt concentration >0.2% at channel 8 alid always, Stop after 5 s when opt concentration <0.2% or 30 s real time.
The sample (approx. 0.25 g) was spread out across the vibri chute, 2 cm from the end to ensure even sample feed.

### Hazards

The work was performed in accordance with the MSDS of the Lactose material (Material ID: 742).

### EXAMPLE 12

### Determination of Water Content for α-Lactose (Monohydrate)

This example describes the procedure for determining the water content of α-lactose by direct addition Karl Fischer titration using a Mitsubishi moisture meter.

### Reagents

Karl Fischer Mitsubishi coulometric reagents:

### Aquamicron AX: Analytical Grade Formamide (4:1)

### Aquamicron CXU

### Instrumental Parameters

### Preparation of the Titration Cell

Typically add a mixture of 120 mL AX reagents and 30 mL Formamide to the anode compartment of the cell. Add 10 mL of CXU reagent to the cathode compartment of the cell

### Use of Nitrogen Purge

Purge dry nitrogen through the anode compartment at approximately 300 mL/min.

### Analysis

Accurately weigh 20 mg±2 mg into a suitable weighing boat. Introduce the sample directly into the titration cell, reweigh the weighing boat to determine the exact sample weight. Record the amount of water present (in μg). Perform the analysis in duplicate.

### Water Content of Weighed Samples

Water (% w/w)=Ww×100/Wu×1000

### Where

Ww=Weight of water detected (μg)
Wu=Weight of sample (mg)

### EXAMPLE 13

### Assay Method and Determination of the Anomeric Purity

### Introduction

This procedure is developed for the determination of the anomeric ratio in α-lactose monohydrate. It is a derivitisation GC method.

### Reagents/Safety

Use reagents that are HPLC grade or other grade of proven suitability. Trimethylsilylimidazole should be stored at 2-8° C.

### Preparation of Solutions

Preparation of Derivitisation Agent

Combine suitable volumes of Pyridine, Trimethylsilylimidazole and Dimethylsulphoxide to obtain a 58.5:22:19.5 mixture. The dissolving solvent is stable for 7 days when stored in a sealed container at 2-8° C.

Preparation of Samples

Weigh accurately 15 mg±0.5 mg of lactose monohydrate into a clean, dry reactivial. Add 4 mL derivitisation solvent and shake for 2 minutes. Leave the sample to derivitise for at least 20 minutes.

All samples must be stored in sealed containers and stored at 2-8° C., the samples are stable for 24 hours.

### Instrumental Parameters

TABLE 6

| Parameter | Typical Value |
|---|---|
| Carrier Gas | Helium |
| Column | DB5-MS 30 m × 0.25 mm i.d. × 1 μm film |
| | thickness |
| Column Head Pressure | 30 p.s.i |
| Split Vent Flow | 50 mL/min |
| Purge Vent Flow | 2 mL/min |
| Injector Temp. | 250° C. |
| Detector Temp. | 300° C. |
| Detector | Flame Ionisation |

TABLE 7
Oven Temperature/Program

| | |
|---|---|
| Initial Temp. | 280° C. |
| Initial Time | 2 minutes |
| Rate | 4° C./min |
| Final Temp. | 300° C. |
| Final Time | 8 minutes |
| Approx. run time | 15 minutes |

### System Suitability

Use the sample preparation described in Example 12 and prepare a sample (which is known to contain both α and β-lactose and ensure it is visually similar to that shown below).

### Typical Retention Times

TABLE 8

| Peak Number | Compound | RT (mins) |
|---|---|---|
| 1 | α-lactose | 9-10 |
| 2 | β-lactose | 10-11 |

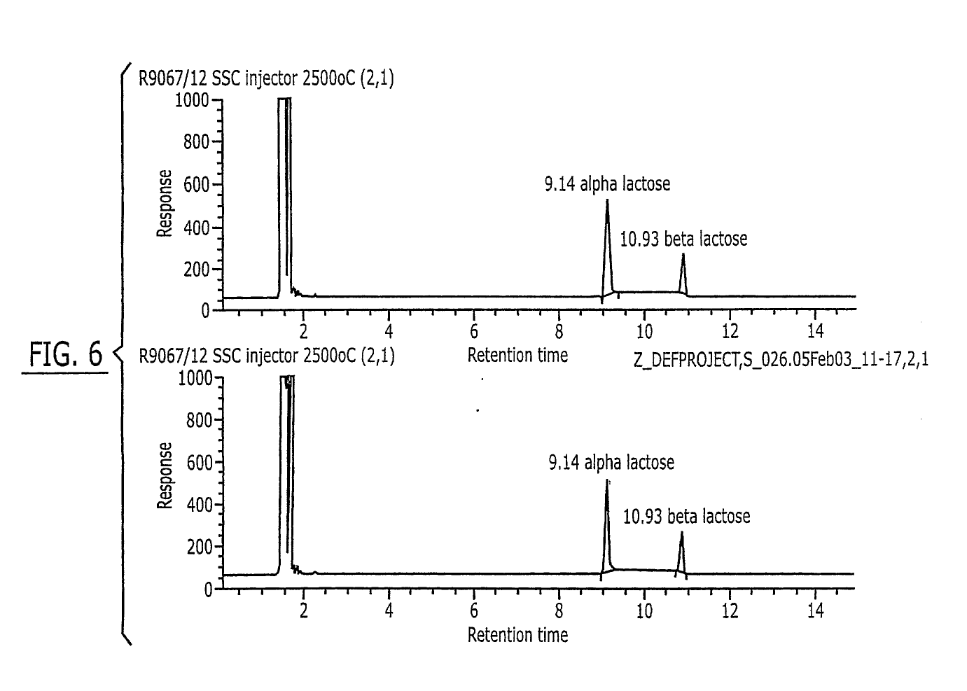

FIG. 6 illustrates the GC results for the two anomers.

### EXAMPLE 14

### Electrical Resistance Tomography Investigation of Mixing

This example illustrates a lab study of the lactose crystallization process carried out on a 3.5 L scale in the electrical resistance tomography reactor. The aim of the study was to evaluate the impact of mixing on the crystallization and generate recommendations for scale-up. Electrical Resistance Tomography (ERT) was used to make sure that a homogeneous suspension was maintained throughout the experiment while using the minimum speed required.

Overall, it was found that it is highly desirable to use a high speed of 120 RPM to maintain good suspension of the crystals in the pilot plant reactor. The impact of shear on the crystals was found negligible.

### Process Parameters

Four experiments were carried out using the following parameters:
Experiment 1: Use of two Viscoprops impellers to obtain scale-up information
Experiment 2: Use of two Viscoprops impellers at high speed
Experiment 3: Use of Retreat Curve Impeller with 1 baffle to check alternative geometry
Experiment 4: Use of two Viscoprops min speed

The viscoprops were selected for the lactose process as they are designed to provide a strong axial flow in viscous solution or slurries thus maintaining good mixing in these systems. They are also quite similar to the impellers used in REACTOR 1. Such a system was operated according to techniques accepted in the art.

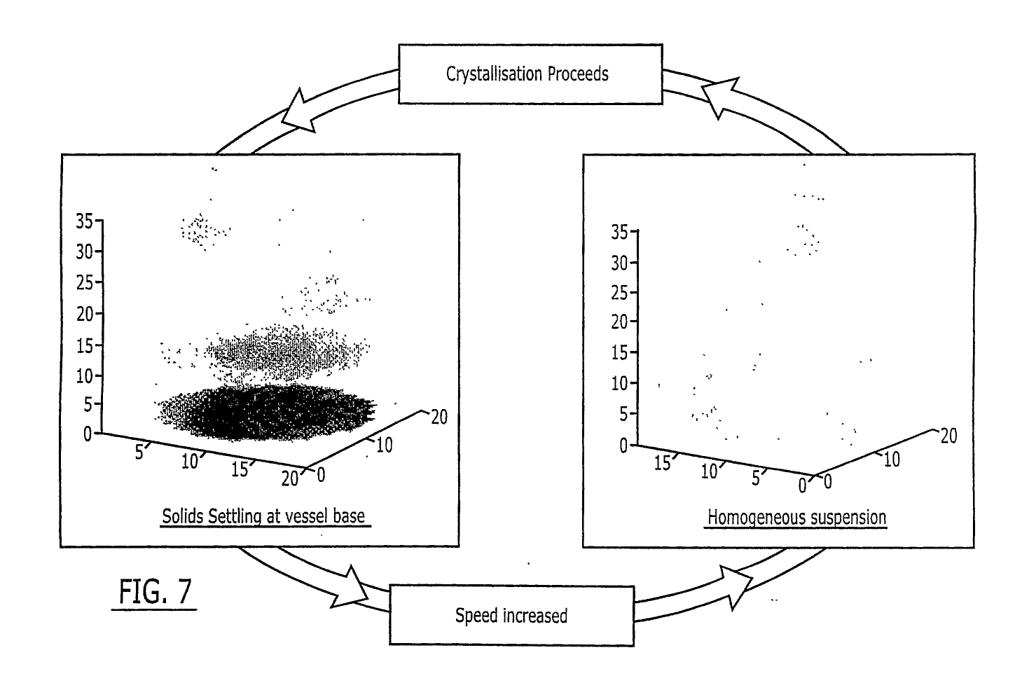

The ERT reactor was applied to the process described herein as the presence of solids in solution is believed to affect the electrical field and can thus be monitored. The technology was applied qualitatively by varying the stirrer speed till an identical conductivity reading was obtained over the 4 planes (equivalent to a homogeneous suspension). FIG. 7 describes the process control applied based on the tomography data.
For the various systems investigated, settling of crystals at the base of the vessel was identified as an important parameter, especially during the initial cooling ramp from 45° C. to 20° C. Any stirrer failure would result in the creation of a solid lump at the base of the vessel, which would be subsequently very difficult to suspend.
The solid lumps can be partially dispersed during the Ostwald ripening.

### EXAMPLE 15

### Equipment Assessment

A CFD evaluation of REACTOR 1 was performed for the batch size of 110 L.

As highlighted, the poor circulation below the impeller may be detrimental to the process on scale-up. However, a scale-up study in a 10 L CLR demonstrated that the crystals were kept mobile by the strong swirling motion due to poor baffling in the vessel. The relatively high heat transfer area to charged volume ratio available in the reactor should help to maintain a homogeneous temperature in the tank and removal of aggregates during Ostwald ripening.

As settling of particles at the base of the reactor was identified as a critical parameter, the just suspension speed (Njs), which is defined as the speed required to prevent settling of particles at the vessel base for more than 2 sec., was selected as a scale-up factor.

On a 3-5 L scale, a stirrer speed of 350 RPM was used to maintain the solids well distributed in the tank without having little if any detrimental effect on the particle size distribution. The Zwietering coefficient can be used for scale-up recommendations under the assumption that liquid and solid properties remain constant on and that geometric similarity is preserved on scale-up. From the lab study carried out, the following equation was then established to estimate the stirrer speed required for the liquid medium in a vessel:

N[RPS]=5.8 RPS(D[m]/0.08 m)^{-0.85}

wherein:

N[RPS] is vessel stirrer speed; and

D[m] is vessel diameter in meters in which the process of the invention occurs. D[m] may encompass various values. For example, in one embodiment, D[m] may range from about 0.01 to about 10 meters.

RPS and m represent revolutions per second and meters respectively.

It is believed that the vessel stirrer speed represented by the above equation may be varied by ±20 percent and still provide acceptable stirring for the process of the invention.

For the purposes of the invention, stirrer speed is believed to significantly impact crystallization. As an example, if the stirrer speed is too slow, settling of solids may occur since the slurry is insufficiently agitated. Conversely, if the stirrer speed is too fast, damage may occur to the solids present in the crystallization slurry.

The above equation is valid for viscoprops or similar geometries.

Filtration and drying trails were carried out on a 10 L scale. Using the scale-up correlation described earlier, a speed of 275 RPM was predicted to be required. However, when carrying out the experiments a speed of 200 RPM was identified as sufficient. Without being bound to theory, such may be attributable to variations in impellar design as the impellars used on a 10 L scale have wider blades than the Viscoprops used on the lab scale, which may improve the pumping capacity of the impellar and the overall efficiency of the set-up. Overall, the correlation tends to overestimate the speed required on scale-up, but such may allow the process to be carried out conservatively.

Using the scale-up correlation described above, a speed of 120 RPM was predicted to be required for REACTOR 1. Using this speed is believed to generate a small increase (approximately 5 percent) in the shear provided to the particles compared to high-speed experiments carried out in the lab. The latter did not seem to have an impact on the particle size distribution. Preferably, experiments should be carried out at a higher speed of 150 RPM, since this is capable of ensuring that homogeneous suspension and improved circulation in the dish base are obtained. Despite the poor circulation below the impeller highlighted in the CFD study, REACTOR 1 is believed to be most suitable at pilot plant for scale-up. The large amounts of solids in the slurry were not capable of being handled by other reactors (conical base) and this could have a dramatic effect on the process.

The above examples are set forth to illustrate the invention. The invention will now be defined by the following claims.

That which is claimed:

1. A process for producing a plurality of lactose particles having a specified particle size distribution, said process comprising:
subjecting a plurality of lactose particles, present in a liquid medium and having a plurality of smaller lactose particles on surfaces of the lactose particles, to conditions such that at least a portion of the smaller lactose particles detach from the plurality of the lactose particles and disperse in the liquid medium;
subjecting the liquid medium to conditions sufficient to cause crystallization to occur on the smaller lactose particle surfaces to form a plurality of larger lactose particles therefrom, wherein a plurality of lactose particles smaller relative to the plurality of larger lactose particles are also present in the liquid medium; and
subjecting the liquid medium to conditions such that at least a portion of the lactose particles smaller relative to the plurality of larger lactose particles are dissolved in the liquid medium, wherein crystallization occurs on the plurality of larger lactose particles.

2. The process according to claim 1, wherein the plurality of lactose particles have a median diameter ranging in size from about 70 microns to about 130 microns.

3. The process according to claim 1, wherein the plurality of smaller lactose particles have a median diameter ranging in size from about 1 micron to about 3 microns.

4. The process according to claim 1, wherein the plurality of larger lactose particles have a median diameter ranging in size from about 20 microns to about 130 microns.

5. The process according to claim 1, wherein the at least a portion of the lactose particles smaller relative to the plurality of larger lactose particles have a median diameter ranging in size from about 1 micron to about 3 microns.

6. The process according to claim 1, wherein the lactose particles formed as a result of the crystallization have a median diameter ranging in size from about 20 microns to about 130 microns.

7. The process according to claim 1, wherein the liquid medium is an aqueous medium.

8. The process according to claim 1, wherein said step subjecting a plurality of lactose particles to conditions such that at least a portion of smaller lactose particles detach from the plurality of the lactose particles comprises the liquid medium being supersaturated with lactose.

9. The process according to claim 1, wherein the detached smaller lactose particles form a homogeneous dispersion.

10. The process according to claim 1, wherein the resulting crystallized lactose particles are substantially free of surface defects.

11. The process according to claim 1, wherein the plurality of lactose particles comprise lactose monohydrate.

12. The process according to claim 11, wherein the plurality of lactose particles comprise alpha lactose monohydrate at an anomeric purity of at least about 97 percent.

13. The process according to claim 1, wherein said step of subjecting a plurality of lactose particles to conditions such that at least a portion of the smaller lactose particles detach from the plurality of the lactose particles occurs at a temperature ranging from about 50° C. to about 70° C.

14. The process according to claim 13, wherein said step of subjecting a plurality of lactose particles to conditions such that at least a portion of the smaller lactose particles detach from the plurality of lactose particles occurs at a temperature of 50° C.

15. The process according to claim 1, wherein said step of subjecting the liquid medium to conditions sufficient to cause crystallization to occur on the smaller lactose particle surfaces to form a plurality of larger lactose particles therefrom occurs at a temperature ranging from about 20° C. to about 50° C.

16. The process according to claim 1, wherein said step of subjecting the liquid medium to conditions such that crystallization occurs on the larger lactose particles occurs at a temperature ranging from about 20° C. to about 70° C.

17. The process according to claim 1, further comprising isolating the resulting crystallized lactose particles from the liquid medium.

18. The process according to claim 17, further comprising drying the resulting crystallized lactose particles.

19. The process according to claim 18, further comprising combining the resulting crystallized lactose particles with at least one medicament to form a pharmaceutical formulation.

20. The process according to claim 19, wherein the pharmaceutical formulation is a dry powder pharmaceutical formulation suitable for inhalation.

21. The process according to claim 19, wherein said at least one medicament is selected from the group consisting of analgesics, anginal preparations, antiinfectives, antihistamines, anti-inflammatories, antitussives, bronchodilators, diuretics, anticholinergics, hormones, xanthines, therapeutic proteins and peptides, salts thereof, esters thereof, solvates thereof, and combinations thereof.

22. The process according to claim 19, wherein the at least one medicament comprises at least one beta agonist.

23. The process according to claim 22, wherein the at least one beta agonist is selected from the group consisting of salbutamol, terbutaline, salmeterol, bitolterol, formoterol, esters thereof, solvates thereof, salts thereof, and combinations thereof.

24. The process according to claim 22, wherein the at least one beta agonist comprises salmeterol xinafoate.

25. The process according to claim 22, wherein the at least one beta agonist comprises salbutamol sulphate.

26. The process according to claim 19, wherein the at least one medicament comprises at least one anti-inflammatory steroid.

27. The process according to claim 26, wherein the at least one anti-inflammatory steroid is selected from the group consisting of mometasone, beclomethasone, budesonide, fluticasone, dexamethasone, flunisolide, triamcinolone, esters thereof, solvates thereof, salts thereof, and combinations thereof.

28. The process according to claim 26, wherein the at least one anti-inflammatory steroid comprises fluticasone propionate.

29. The process according to claim 19, wherein the at least one medicament comprises at least one beta agonist and at least one anti-inflammatory steroid.

30. The process according to claim 29, wherein the at least one beta agonist comprises salmeterol xinafoate and the at least one anti-inflammatory steroid comprises fluticasone propionate.

31. The process according to claim 19, wherein the at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipratropium, phenylephrine, phenylpropanolamine, pirbuterol, reproterol, rimiterol, isoetharine, tulobuterol, (−)-4-amino-3,5-dichloro-α-[[[6-[2-(2-pyridinyl)ethoxy]hexyl]methyl]benzenemethanol, esters thereof, solvates thereof, salts thereof, and combinations thereof.

32. The process according to claim 19, wherein the at least one medicament is selected from the group consisting of albuterol sulphate, salmeterol xinafoate, fluticasone propionate, beclomethasone dipropionate, and combinations thereof.

33. The process according to claim 19, wherein said pharmaceutical formulation further comprises at least one additional excipient.

34. The process according to claim 1, wherein said process occurs in a vessel.

35. The process according to claim 34, wherein the aqueous medium is subjected to agitation by a stirrer, wherein the speed of the stirrer is determined by the equation:
N[RPS]=5.8 RPS(D[m]/0.08 m)^{-0.85}±20 percent
wherein:
N[RPS] is vessel stirrer speed;
D[m] is vessel diameter; and
RPS and m represent revolutions per second and meters respectively.

36. The process according to claim 35, wherein D[m] ranges from about 0.01 to about 10 meters.
